(19) United States
(12) Patent Application Publication
(10) Pub. No.: US 2008/0125653 A1
(43) Pub. Date: May 29, 2008

(54) DENSITY AND POROSITY MEASUREMENTS BY ULTRASOUND

(75) Inventors: Peter P. Antich, Richardson, TX (US); Charles Y. C. Pak, Dallas, TX (US)

Correspondence Address: CHALKER FLORES, LLP, 2711 LBJ FRWY, Suite 1036, DALLAS, TX, 75234, US

(73) Assignee: Board Of Regents, The University Of Texas System, Austin, TX (US)

(21) Appl. No.: 11/563,499

(22) Filed: Nov. 27, 2006

(51) Int. Cl. A61B 8/00

(52) U.S. Cl. 600/438; 600/437

(57) ABSTRACT

The present invention is an apparatus, method and system for determining cancellous or cortical bone density, cortical bone thickness, bone strength, bone fracture risk, bone architecture and bone quality by acoustically coupling an ultrasound transducer to nearby skin over a bone, reflecting one or more pulses produced by the ultrasound transducer from the bone, and detecting the reflected pulse reflected by the bone, wherein bone porosity and other properties are calculated at a low frequency, a high frequency or both a low and a high frequency.

### TECHNICAL FIELD OF THE INVENTION

The present invention relates in general to the field of material analysis, and more particularly, to novel devices, methods and systems for the determination of density, porosity and thickness of bone by ultrasound.

### BACKGROUND OF THE INVENTION

Without limiting the scope of the invention, its background is described in connection with bone density measurements, as an example.

Need for Non-Invasive Measurement of Bone Strength. Osteoporosis is a major medical problem, with a large percentage of elderly persons being susceptible to sustain non-traumatic fractures (bone fractures from minimum trauma). Bone strength is a primary predictor of bone fractures. Bone strength is determined by bone density and bone quality, as well as by other factors such as thickness.

Currently, bone density can be measured by several methods, including: dual energy x-ray absorptiometry, computer-assisted tomography and transmission ultrasound. The first method measures “bone mineral density” since they estimate the amount of bone mineral in a given bone tissue. From epidemiological studies, bone mineral density is inversely correlated with the rate of skeletal fractures. Thus, bone mineral density has been used to define osteoporosis, with a value below 75% of normal peak value is referred to as osteoporosis even in the absence of fractures. CT measurements are more closely associated with density while transmission ultrasound absorptiometry is a correlate of bone mineral density.

Recent discoveries, however, have presented situations in which bone mineral density may be dissociated from bone quality. Introduced in 1996, a new class of drugs called “bisphosphonate” has been widely used for the treatment of osteoporosis (Liberman et al., N. Engl. J. Med. 333:1437-1443, 1995). With long-term use, new studies suggest that these drugs can severely impair bone quality leading to recurrent fractures that do not heal properly despite increased bone mineral density (Ott, J. Clin. Endo. Metab. 86:1835, 2001; Odvina, et al., J. Clin Endo Metab, 90:1294-1301, 2005; Richer et al., Osteop Int, 16:1384-1392, 2005; Li et al., Calc. Tissue Intern. 69:281-286, 2001). Moreover, with improvement in surgical techniques and in medical treatments to prevent rejection, more patients are living longer after kidney (renal) transplantation. These patients are known to have increased susceptibility to fractures, since they probably have defective bone from taking steroids and suffer from other factors that are harmful to bone.

The inventors of the current application have previously filed a patent for a device that can measure reliably, quickly and non-invasively the quality of bone in vivo, from reflected ultrasound at the critical angle. Using this device, material elasticity of bone was shown to be substantially reduced in aforementioned conditions of long-term bisphosphonate treatment and renal transplantation (Richer et al., Osteop Int, 16:1384-1392, 2005), suggesting that intrinsic bone quality was impaired.

### SUMMARY OF THE INVENTION

The current invention includes novel devices and method for measuring porosity of cortical and cancellous bone (from which “true” or apparent bone density can be derived), cortical bone thickness, and degree of bone mineralization, by using broader overall principles of critical angle reflectometry. Combined with material elasticity obtained by the same reflected ultrasound method, these newly derived measurements can be used to estimate bone strength.

The apparatus, method and system of the present invention use ultrasound reflectometry to improve patient care by providing bone density and mineralization of cortical and cancellous bone, as well as cortical bone thickness, by using a non-invasive, rapid and reliable method based on ultrasound critical angle relectometry. Combined with material elasticity from reflected ultrasound, the aforementioned bone properties can be used to estimate bone strength.

The present invention includes devices, methods and systems for measuring density of cancellous or cortical bone, degree of bone mineralization and cortical bone thickness, from which bone strength, fracture risk, and architecture may be estimated. By acoustically coupling an ultrasound transducer to nearby skin over a bone, reflecting one or more pulses produced by the ultrasound transducer from the bone and by detecting the reflected pulse reflected by the bone, the porosity of bone can be calculated at a low frequency, a high frequency or both a low and a high frequency, or multiple frequencies. A calculated density can be derived from porosity. The transducer may be selected from a focused or a planar transducer and the transducer may be positioned such that the reflection of the pulse is detected at various angles to improve the calculation of the bone density. Examples of frequencies that may be used include a low frequency pulse is between 0 Hz and 3.5 MHz. A high frequency pulse is generally at or above 3.5 MHz.

Multiple measurements of the bone density at low frequency are used to determine the extent of holes porosity) that are found in the bone. Multiple measurements of bone density at a high frequency are used to determine the extent of holes (porosity) that are found in the bone as well as the degree of bone mineralization. The target bone may be any bone in a body, e.g., a long bone of the arm or leg, hip, spine. The reflection may be measured at a large angle, for example, the large angle of between 60 and 120 degrees or between 85 and 95 degrees.

The invention purports to a device for measuring cancellous bone density that includes an ultrasound transducer capable of sending pulses at two or more frequencies, wherein the transducer is acoustically coupled to a bone target; one or more ultrasound pulse detectors positioned to detect one or more pulses reflected from the bone target, wherein bone density is calculated at a low frequency, a high frequency or both a low and a high frequency (e.g., at multiple frequencies); and a processor capable of calculating a bone density based on the detected reflections to determine bone density. The array may be positioned at a large angle of between 60 and 120 degrees, e.g., at between 85 and 95 degrees.

The invention includes a method of measuring cortical bone thickness by acoustically coupling an ultrasound transducer to nearby skin over a bone at an angle; reflecting one or more pulses produced by the ultrasound transducer along the length of the bone; detecting the reflected pulse reflected by the bone using a linear array of receivers disposed downstream from the ultrasound transducer, wherein the thickness of cortical bone is calculated from location and time of the signals reflected from within the cortical bone layer at different points along the length of the array. Multiple measurements of the bone density at low frequency may be used to determine the extent of holes that are found (porosity) in the bone. Multiple measurements of the bone density taken at high frequency are used to determine the extent of holes that are found in the bone as well as degree of mineralization.

A device for measuring cortical bone thickness may also include an ultrasound transducer acoustically coupled to a bone target at an angle; and a linear array of receivers disposed downstream from the ultrasound transducer, wherein one or more pulses produced by the ultrasound transducer reflected at different points along the length of the bone are used to calculate the thickness of cortical bone density based on the frequency and strength of the reflection.

### BRIEF DESCRIPTION OF THE DRAWINGS

For a more complete understanding of the features and advantages of the present invention, reference is now made to the detailed description of the invention along with the accompanying figures and in which:

FIGS. 1A and 1B shows that cancellous bone is a two phase material;

FIG. 2 shows one embodiment of the reflected ultrasound method of the present invention;

FIG. 3 is a graph that shows the results of the estimated porosity and the calculated density of all the samples;

FIG. 4 is a graph that shows the results of the in vitro study;

FIG. 5 is a graph that shows an inverse linear relationship between the average porosity and the peak amplitude of the reflected ultrasound signal;

FIG. 6 shows the basic calculations of reflective ultrasound calculations;

FIG. 7 shows a cortical bone thickness ultrasound detector (10) that may be used to detect critical architectural features of a bone;

FIG. 8 is a graph that shows the dependence of apparent bone density results on thickness;

FIG. 9 is a graph that shows another array configuration of the present invention that is used to detect bone density using large-angle scattering.

FIG. 10 shows the bimodal distribution at different frequencies using the present invention;

Note that the transmitter has been replaced with an interchangeable piezoelectric element operating typically at a frequency of 3.5 MHz or higher.

FIG. 10 shows that a measurement of UCR velocity using the new configuration at 5 MHz.

FIGS. 11 and 12 are graphs that show inverse relationship between the reflected signal (peak-to-peak amplitude, FIG. 11 and integrated amplitude, FIG. 12) and porosity at 5 MHz; These graphs show that the measurement is affected by bone architecture, in particular that there are differences between the amplitudes measured along different faces of the sample.

FIG. 12 is a graph that shows inverse relationship between the averaged (over faces) integral of the reflected signal and porosity at 5 MHz;

FIG. 13 is a graph that shows inverse relationship between average peak-to-peak amplitude Vpp of the reflected signal and porosity at 5 MHz;

FIG. 14 is a graph that shows inverse relationship between the averaged integrated amplitude of the reflected signal and porosity at 5 MHz in which the power spectrum of the signal measured at 90 degrees from incidence was measured.

FIG. 15 is a graph that shows direct relationship between integrated low frequency band in the power spectrum and porosity

FIG. 16 is a graph that shows inverse relationship between integrated high frequency band in the power spectrum for large-angle scattering and porosity at a high frequency (5 MHz).

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

As used herein, the term “true bone density,” “apparent bone density” and “calculated bone density” are used interchangeably to refer to amount of bone material in a given volume of bone tissue, where the bone material includes mineral phase (calcium phosphate), bone matrix (collagen) and bone marrow. The above terms are to be distinguished from “bone mineral density”, which refer to the amount of bone mineral in a given volume of bone tissue. Cancellous bone is known to have several primary characteristics, including: thickness, degree of bone mineralization, material elasticity, pore size, pore volume, pore shape and combinations thereof.

As used the term “emitting” is used to describe the transmission of an ultrasound wave or pulse by an ultrasound wave transmitter. As used herein the term “receiving” is used to describe the reception by an ultrasound wave receiver of an ultrasound pressure pulse or wave reflected by a material. Together the transmitter and the receiver are described as forming a “transducer” that is able to emit and receive an ultrasound wave reflected from a target material, whether the wave hits the target directly and/or if the wave traverses an ultrasound conductive or transmissive material prior to striking the target, which may be a target point or plane.

As used herein, the term “reflectrometry” is used to describe the reflection an ultrasound wave emitted from an ultrasound transmitter after striking a target, where the reflected ultrasound wave travels back toward the ultrasound transmitter or reflects at a large angle as compared to the position of the transmitter. Reflectometry may be contrasted with transmission ultrasound detection, wherein the ultrasound wave travels through the target (like an X-ray) and the ultrasound wave is detected at about 180° from the transmitted. In order to receive or detect an ultrasound wave reflected from a target as an ultracritical reflection, the receivers of the ultrasound wave are at, behind or about the ultrasound transmitter, e.g., in the direction of the ultracritical reflection, which is generally the normal of the transmitter and the receivers.

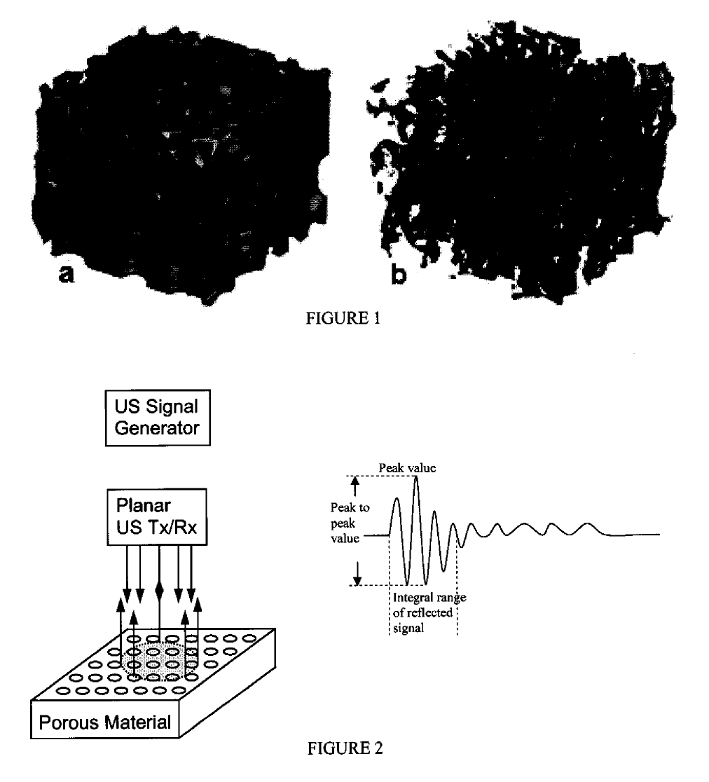

FIGS. 1A and 1B shows that cancellous bone is a two phase material. FIG. 1A shows the two phases of a cancellous bone, trabeculae and plates as well as the fatty marrow and the pores. The pore walls are made of the calcified materials of trabeculae and plates, and fatty marrow is found within the pores. The porosity of cancellous bone changes rapidly with metabolic and disease status. Previous research indicates that in osteoporosis, plates and trabeculae become thinner and gradually disappear; as a result, the porosity increases and bone material properties changes. FIG. 1B shows bones with osteoporosis, the plates and the trabeculae become thinner and fragile causing the bone to be more likely to break. In osteoporosis the porosity increases and the bone material properties change. Therefore, it would be a great advantage in detecting osteoporosis and assessing treatment to monitor porosity quantitatively.

FIG. 2 shows the reflected ultrasound method of the present invention. Devices and methods that use reflected ultrasound to detect porosity are disclosed. In operation, an ultrasound signal is generated and transmitted by a planar ultrasound transmitter. As the ultrasound wave (e.g., a pulse) strikes the target, the ultrasound signal is at least partly reflected back from the porous material. The reflected signals are received by the ultrasound receiver and then recorded for further analysis. To reveal the relationship between the reflected ultrasound signal and the material's porosity, a computer simulation was first conduced to give a theoretic prediction.

In the computer simulation, the field II ultrasound generator, which is running under MATLAB, was used to simulate the ultrasound generator, transmitter and receiver. Four porous phantoms were used to simulate different porosity. The computer simulation shows that for the ideal case there is a linear relationship between a material's porosity and the peak amplitude of the reflected signal.

Next, the computer simulation was compared to a study using target phantoms. Four phantoms of the same size with high density plastics with different porosities were fabricated. The phantoms were immersed in water to simulate the soft tissues overlying bone tissue in vivo. A 5 MHz planar ultrasound transducer was used. The peak value and the integral of the reflected signals were analyzed. The fabricated phantoms were made from acrylic plastic with dimensions of 2 cm×2 cm×0.6 cm and 4 different porosities. Using the phantoms, there was an inverse linear relationship between the porosity and the parameters of the reflected ultrasound signal. The results of the phantom study agreed with computer simulation.

Next, an in vitro bone sample study was conducted. Twelve cancellous bone samples were cut in 1×1 inch cubes from cow femur bones. These bones were immersed in alcohol for two weeks and defatted. The porosities of these bone samples were estimated by calculating the ratio of the mass in air to the “wetted mass” when the sample is immersed in water and all the air is drained from the pores. The apparent density was defined as the ratio of the weight of dry mass over the total volume.

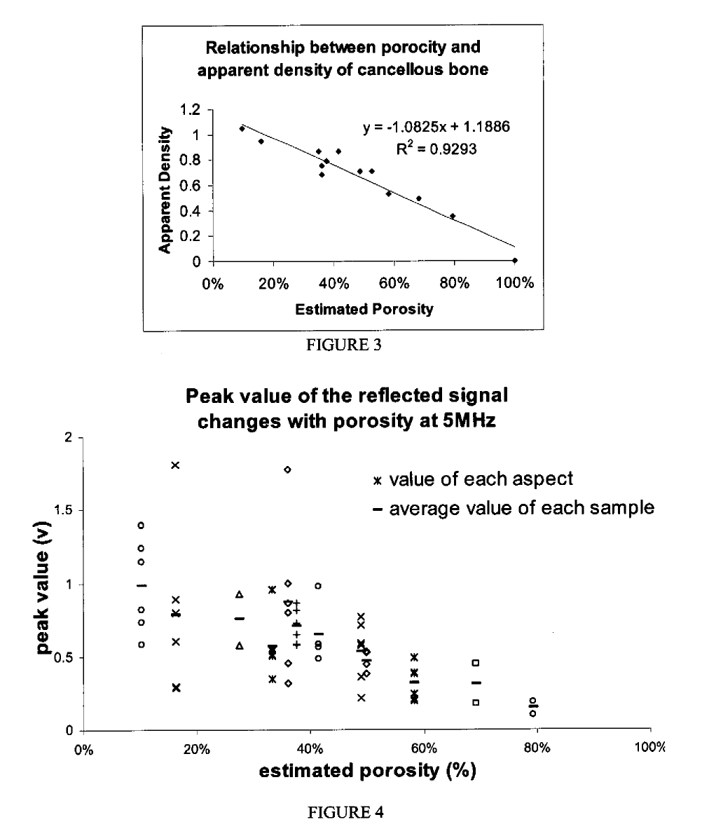

FIG. 3 is a graph that shows the results of the estimated porosity and the apparent density of all the samples. It was found that the apparent density is inversely and linearly related to the porosity.

FIG. 4 is a graph that shows the results of the in vitro study. In FIG. 4, the peak values of the reflected signal from different faces of each sample were plotted. The plot shows that the observed porosity depends upon the face interrogated, showing heterogeneity of the porosity. Since the reflected signal from different faces of one single bone sample may vary substantially in agreement with changes in architecture, the average the values for each sample was used for the over-all porosity.

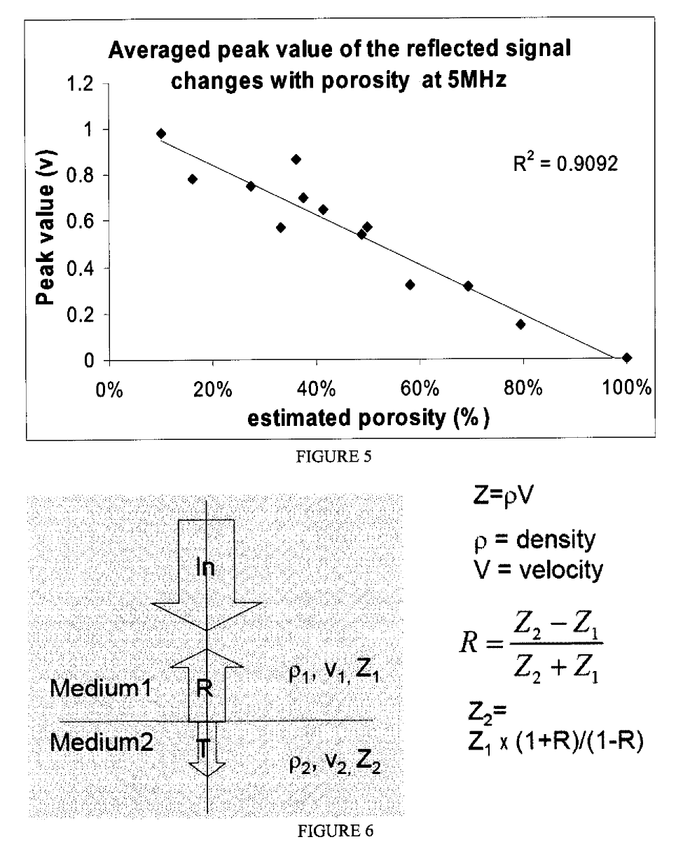

FIG. 5 is a graph that shows a linear inverse relationship between the average porosity and the peak amplitude of the reflected ultrasound signal. The average porosity is thus correlated with the density, while the local porosity depends upon the heterogeneity of the cancellous bone. Using reflective ultrasound the average porosity of cancellous bone can be directly determined by the parameters of the ultrasound signals reflected from the bone, as a linear inverse relationship between them. It is also demonstrated herein that the observed porosity depends upon the face interrogated which shows heterogeneity of the porosity. This orientation dependent technique may be used to monitor not only the density of cancellous bone, but also effect of the microarchitecture.

FIG. 6 shows the basic calculations of reflection ultrasound calculations. The quantity measured by ultrasound in back-reflection is the acoustic impedance. The density is the impedance divided by the velocity V, where V is measured by ultracritical reflectometry (UCR). R in a single reflection cannot be measured with a high precision. To have satisfactory precision, multiecho reflections from interface between buffer and the material may be used to increase the precision of the analysis, basically following the equation: V=R+R2+R3+

Table 1 shows a group of materials tested using multiecho multiple reflection ultrasound reflectometry. Density so calculated in plastics and high density acrylate (HDPL) corresponded closely with the directly measured values.

TABLE 1
Multiecho multiple reflection ultrasound reflectometry

| | Directly Measured | Calculated density from |
|---|---|---|
| Material | Density(g/cm³) | Experiment (g/cm³) |
| Steel | 7.606 | — |
| Water | 1.0 | — |
| Plastics | 1.417 | 1.455 |
| HDPL | 1.164 | 1.158 |

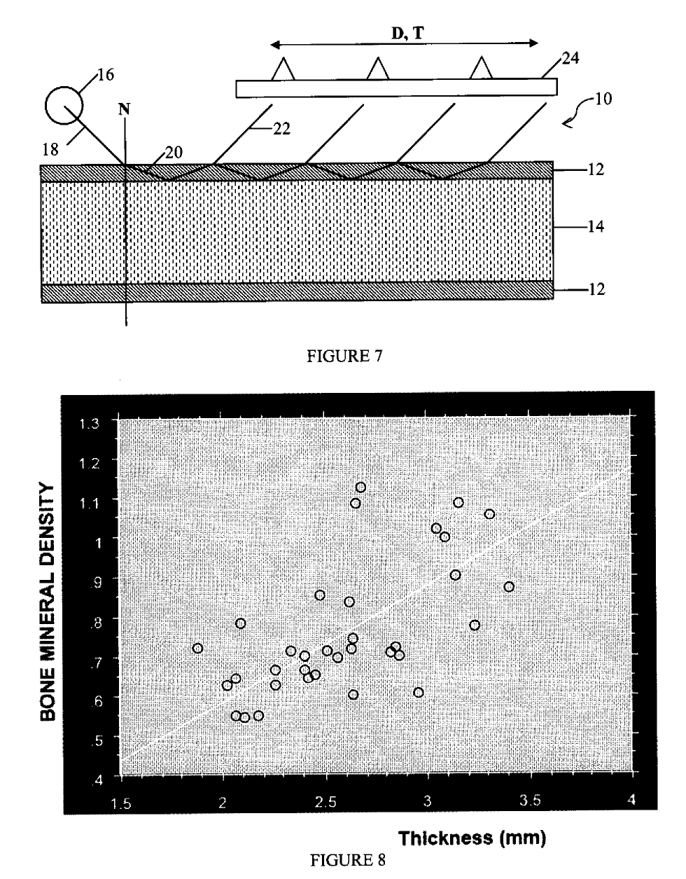

FIG. 7 shows a cortical bone thickness ultrasound detector (10) that may be used to detect critical architectural features of a bone. Depicted is a cross sectional view of a cortical bone (12) and a trabecular bone (14) positioned as a target for an ultrasound source (16). An ultrasound wave (18) is transmitted toward the cortical bone (12) at an angle other than the Normal (N) and changes its angle as it enters the cortical bone (12) and reflects off the interface with the trabecular bone (14), shown as wave (20). The reflected wave (22) exits the cortical bone (12) and is detected with a receiver array (24). The receiver array (24) is used to calculate density, velocity and thickness with a single device. The features of the material can be measured using the present invention, specifically, thickness is measured by detecting the ultrasound along distance (D) (location of receiver element) from the ultrasound source (16) and time (T) (time of arrival at element). The location and time required for the wave to enter and exit the cortical bone based on a defined or known angle between the ultrasound source and the cortical bone (12) will depend upon the thickness of the cortical bone (12) and the velocity. The two quantities can be calculated independently, using the relationships V Sin θ=constant and D=thickness×tan θ.

FIG. 8 is a graph that shows the bone mineral density results measured radiologically are directly dependent on thickness measured using the device depicted in FIG. 7.

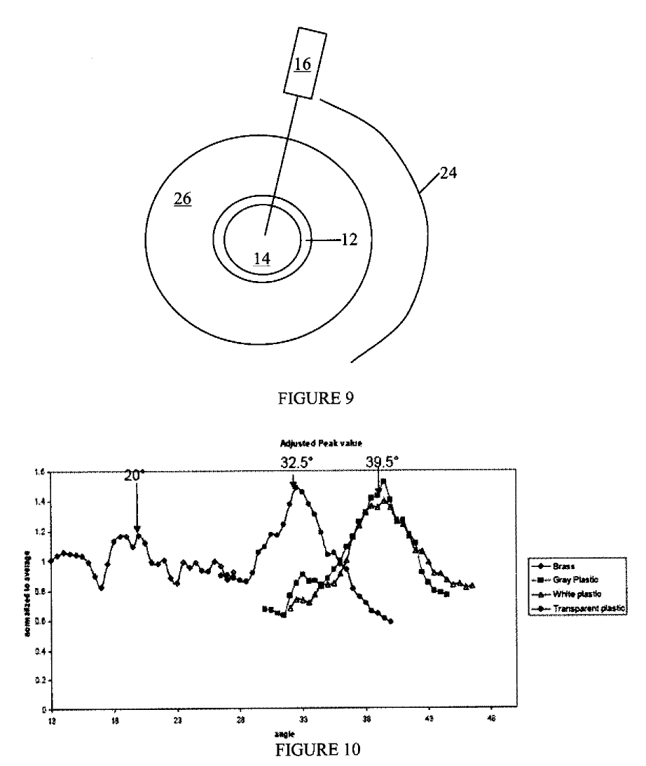

FIG. 9 is a graph that shows a scheme that is used to detect bone density using large-angle scattering. Briefly, an ultrasound source (16) is positioned to target a cortical bone (12) and trabecular bone (14) within a tissue (26). The reflections from the bone (12, 14) are detected at an array (24). The ultrasound source (16) in this embodiment is capable of transmitting ultrasound waves with two or more wavelengths.

FIG. 10 shows UCR spectra obtained with the new UCR configuration, showing that it can be used to measure small sample or biopsy properties.

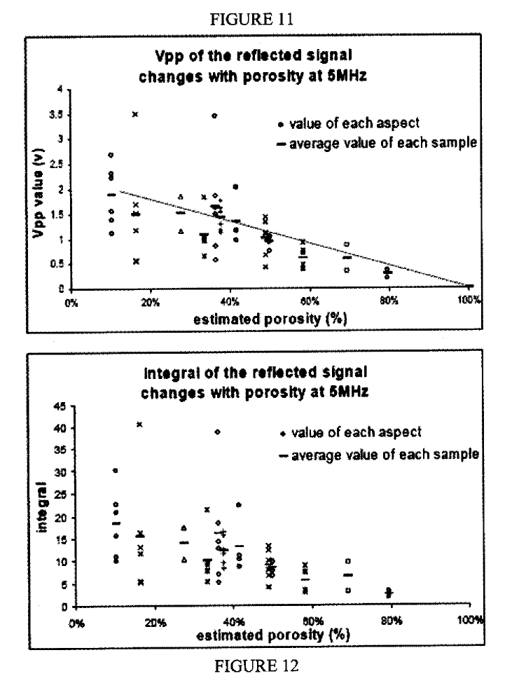

FIG. 11 is a graph that shows the poorly defined inverse relationship between the peak-to-peak amplitude of the reflected signal and porosity at 5 MHz for different faces of a bone sample.

FIG. 12 is a graph that shows the poorly defined inverse relationship between the averaged integral of the reflected signal amplitude and porosity at 5 MHz.

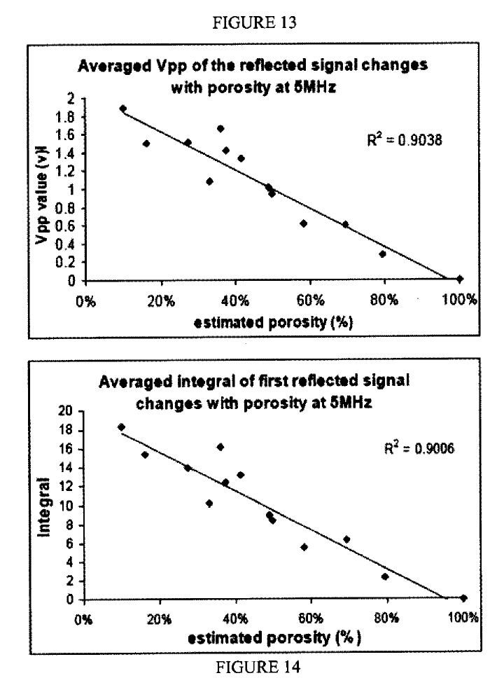

FIG. 13 is a graph that shows the stronger inverse relationship between the average peak-to-peak amplitude (averaged over faces) of the reflected signal and porosity at 5 MHz.

FIG. 14 is a graph that shows the stronger inverse relationship between the integral of the amplitude (averaged over faces) of the reflected signal and porosity at 5 MHz.

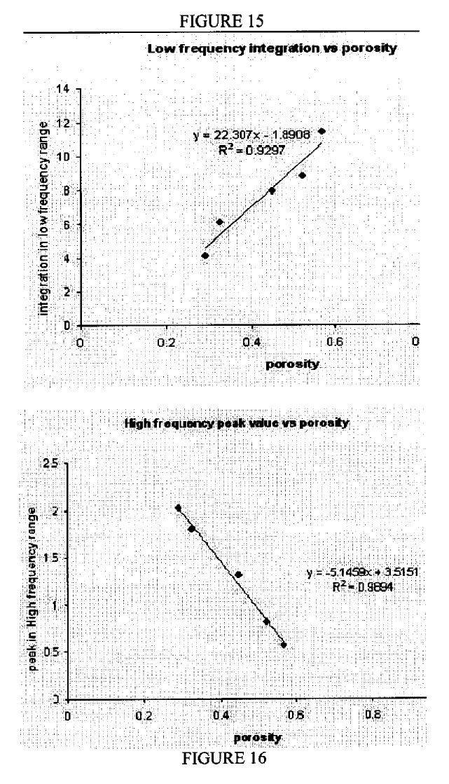

FIG. 15 is a graph that shows the low-frequency integral of the power spectrum of the reflection from a bone at a large angle. The low frequency peak is dependent upon porosity: that is, the reflections are linear and proportional to porosity.

FIG. 16 is a graph that shows inverse relationship between porosity and high frequency component of the power spectrum.

Material and Method for Porosity Study by Pulse-echo Ultrasound.

Computer Simulation. To reveal the relationship between the reflected ultrasound signal and the material's porosity, a computer simulation was first conducted to give a theoretic prediction. The computer simulation was programmed using MATLAB® (The Mathworks, Natick, Mass.). Four porous phantoms were made to simulate different porosity (FIG. 1). The ultrasound generator, transmitter and receiver were simulated by calling the corresponding functions from the Field II ultrasound simulation program (copyrighted freeware by Jørgen Arendt Jensen, Denmark). The simulated transducer and receiver were planar PCT transducers with the central frequency of 5 MHz. The simulation program mimicked the process of ultrasound wave interacting with the porous phantom and calculated the reflected ultrasound signal automatically.

Phantom Study. Four phantoms of the same size (2 cm×2 cm×0.6 cm) were made with acrylic plastics, and fabricated with the porosity of 0%, 14%, 25% and 49%, respectively. These phantoms were immersed in water, and held parallel to the transducer surface by a home-made phantom holder.

The ultrasound signals were generated by an ultrasound pulser/receiver (model 5052PR, PANAMETRICS, Inc., Waltham, Mass.). The pulser/receiver was executing at the pulse-echo mode. No attenuation, high-pass filter and damping were applied to the generated signal, while there was a 40 dB gain applied to the received signal. A planar PCT transducer (V309, PANAMETRICS, Inc., Waltham, Mass.) with the central frequency of 5 MHz was used as the transmitter/receiver. The pulser/receiver was connected to a digital oscilloscope (2430A, Tektronix, Inc., Beaverton, Oreg.), where the real-time received signal was displayed. The oscilloscope was then connected to the computer via a PCI-GPIB IEEE 488.2 Card and Cable (National Instruments Corp., Austin, Tex.), which allowed the loading of the displayed signal from the oscilloscope to the computer. The data analysis was performed in the LabVIEW™ (National Instruments Corp., Austin, Tex.) environment.

in vitro Bone Sample Study. Twelve cancellous bone samples were cut from bovine femur by a 7.5″ power band saw (Black & Decker Corp., Towson, Md.). The samples were collected from the head of the femur, greater trochanter and condyles; due to the irregular shape of the sites, the bone samples were cut into different sizes from 1″×0.5″×0.5″ to 1″×1″×1″. These bone samples were immersed in 70% ethanol for two weeks and defatted.

The porosities of these bone samples were estimated by calculating the weight difference between the dry sample in air and the “wetted sample” when it is immersed in water and all the pores are saturated with water, as given below:

$$porosity = \frac{\begin{matrix}weight of “ wetted mass ” - \\ weight of dry mass\end{matrix}}{density of water * volume of the sample} * 100 \% \qquad \text{(Eq. 1)}$$

The apparent density was defined as the ratio of the weight of dry mass over the total volume:

$$Apparent density = \frac{dry weight}{total Volume} \qquad \text{(Eq. 2)}$$

The setup for the bone sample study was exactly the same as the phantom study.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES

J. A. Jensen: Field: A Program for Simulating Ultrasound Systems, Paper presented at the 10th Nordic-Baltic Conference on Biomedical Imaging Published in Medical & Biological Engineering & Computing, pp. 351-353, Volume 34, Supplement 1, Part 1, 1996.
Parfitt A. M., “Trabecular bone architecture in the pathogenesis and prevention of fracture”, American Journal of Medicine, 82(1B): 68-72, 1987.

What is claimed is:

1. A method of measuring cancellous or cortical apparent bone density, bone strength, bone fracture risk, bone architecture and bone quality comprising the steps of:
acoustically coupling an ultrasound transducer to nearby skin over a bone;
reflecting one or more pulses produced by the ultrasound transducer from the bone; and
detecting the reflected pulse reflected by the bone, wherein bone density is calculated at a low frequency, a high frequency or both a low and a high frequency.

2. The method of claim 1, wherein the transduced pulse is selected from a focused or a planar transducer.

3. The method of claim 1, wherein the reflection of the pulse is detected at various angles to improve the calculation of the bone density.

4. The method of claim 1, wherein a low frequency pulse is between 0 Hz and 3.5 MHz.

5. The method of claim 1, wherein a high frequency pulse is above 3.5 MHz.

6. The method of claim 1, wherein multiple measurement of the bone density at low frequency are used to determine the extent of holes that are found in the bone.

7. The method of claim 1, wherein multiple measurement of the bone density at high frequency are used to determine the extent of bone porosity as well as mineralization in the bone.

8. The method of claim 1, wherein the bone is a long bone of the arm or leg.

9. The method of claim 1, wherein the reflection is measured at a large angle.

10. The method of claim 1, wherein the reflection is measured at a large angle of between 60 and 120 degrees.

11. The method of claim 1, wherein the reflection is measured at between 85 and 95 degrees.

12. A device for measuring cancellous or cortical bone density comprising:
an ultrasound transducer capable of sending pulses at two or more frequencies, wherein the transducer is acoustically coupled to a bone target;
one or more ultrasound pulse detectors positioned to detect one or more pulses reflected from the bone target, wherein bone density is calculated at a low frequency, a high frequency or both a low and a high frequency; and
a processor capable of calculating a bone density from the detected reflections.

13. The device of claim 12, wherein the array is positioned at a large angle of between 60 and 120 degrees.

14. The device of claim 12, wherein the array is positioned at between 85 and 95 degrees.

15. A method of measuring cortical bone thickness comprising the steps of:
acoustically coupling an ultrasound transducer to nearby skin over a bone at an angle;
reflecting one or more pulses produced by the ultrasound transducer along the length of the bone; and
detecting the reflected pulse reflected by the bone using a linear array of receivers disposed downstream from the ultrasound transducer, wherein the thickness of cortical bone density is calculated based on the frequency and strength of the reflections by measuring the signals reflected from within the cortical bone layer at different points along the length of the array.

16. The method of claim 15, wherein the transduced pulse is selected from a focused or a planar transducer.

17. The method of claim 15, wherein the reflection of the pulse is detected at various angles to improve the calculation of the cortical bone thickness.

18. The method of claim 15, wherein a low frequency pulse is between 0 Hz and 3.5 MHz.

19. The method of claim 15, wherein a high frequency pulse is above 3.5 MHz.

20. The method of claim 15, wherein multiple measurement of the bone density at low frequency are used to determine the extent of holes that are found in the bone.

21. The method of claim 15, wherein multiple measurement of the bone density at high frequency are used to determine the extent of holes that are found in the bone.

22. The method of claim 15, wherein multiple measurement of the bone density at high frequency are used to determine the degree of mineralization of the bone.

23. The method of claim 15, wherein the bone is a long bone of the arm or leg.

24. A device for measuring cortical bone thickness comprising:
an ultrasound transducer acoustically coupled to a bone target at an angle; and
a linear array of receivers disposed downstream from the ultrasound transducer, wherein one or more pulses produced by the ultrasound transducer reflected at different points along the length of the bone are used to calculate the thickness of cortical bone density based on the frequency and strength of the reflection.
